(19) United States
(12) Patent Application Publication
(10) Pub. No.: US 2020/0265919 A1
(43) Pub. Date: Aug. 20, 2020

(54) ANALYZING DEVICE, ANALYSIS METHOD PROGRAM, AND NON-VOLATILE STORAGE MEDIUM

(71) Applicant: National Institute of Advanced Industrial Science and Technology, Tokyo (JP)

(72) Inventors: Katsuhisa Horimoto, Tokyo (JP); Kazuhiko Fukui, Tokyo (JP); Harumi Kagiwada, Tokyo (JP)

(73) Assignee: National Institute of Advanced Industrial Science and Technology, Tokyo (JP)

(21) Appl. No.: 16/761,165

(22) PCT Filed: Nov. 5, 2018
(86) PCT No.: PCT/JP2018/040960

§ 371 Date: May 1, 2020

(30) Foreign Application Priority Data
Nov. 7, 2017 (JP) 2017-215038

(51) Int. Cl. G16B 5/20; G06N 7/00; G01N 33/68

(52) U.S. Cl. CPC G16B 5/20; G01N 33/6845; G06N 7/005

(57) ABSTRACT

An analysis method for using an analyzer to analyze consistency between data on the amount of reaction obtained by a predetermined treatment performed on a plurality of substances included in a specimen and a known pathway includes: the analyzer acquiring the data on the amount of reaction for a plurality of specimens; and the analyzer reading, from a storage stored with data on a known pathway including the substances as nodes, the data on the known pathway and determining consistency between the known pathway and the data on the amount of reaction. The known pathway is an undirected graph.

### TECHNICAL FIELD

The present invention relates to a technique to analyze consistency between data on the amount of reaction obtained by a predetermined treatment performed on a plurality of substances included in a specimen and a known pathway.

### BACKGROUND ART

Protein phosphorylation is a kinase-catalyzed reaction, and regulates various vital functions including cell proliferation, transcriptional control, cell death, and metabolism. Cancer and other diseases, too, are known to be caused by abnormalities in phosphorylating enzymes, and phosphorylation signaling pathways have a profound effect also on human diseases. Analysis of phosphorylation signaling is considered to yield useful information on drug discovery, diagnosis of diseases, and the like (Non-patent document 1).

Non-patent document 1 discloses a method of analyzing phosphorylation signals using a peptide array. The method in this document uses a comparison between phosphorylation patterns of normal cells and those of specimen cells for early diagnosis of diseases (Non-patent document 1, the right side on page 99 to the left side on page 100).

### PRIOR ART DOCUMENTS

### Non-Patent Documents

Non-patent document 1: Takahiro FUNATSU et al., Development of titanium oxide plate immobilized substrate peptide for detection of protein kinase activity, Research Report of Kitakyushu National College of Technology, 44, 2011-01
Non-patent document 2: Katsuhisa HORIMOTO et al., Network evaluation from consistency of the graph structure with the measured data, BMC Systems Biology 2008, 2: 84 (Oct. 1, 2008)

### SUMMARY OF THE INVENTION

### Problems to be Solved by the Invention

The method described in Non-patent document 1 could only find peptides with different degrees of phosphorylation between specimen cells and normal cells, and it could not analyze their phosphorylation signaling pathways.

One of the present inventors published a method of evaluating consistency between measured data and a network structure in Non-patent document 2. The method disclosed in Non-patent document 2 is for when the network structure is a directed acyclic graph (DAG). There has been no document that discloses a method of evaluating consistency between a network structure of an undirected graph (non-DAG) and measured data.

A purpose of the invention made in the foregoing background is to provide a method of analyzing consistency between data on the amount of reaction of a plurality of substances included in a specimen and a known pathway.

### Means for Solving the Problems

An analysis method of the invention is for using an analyzer to analyze consistency between data on the amount of reaction obtained by a predetermined treatment performed on a plurality of substances included in a specimen and a known pathway, and the analysis method comprises the steps of:
(a) the analyzer acquiring the data on the amount of reaction for a plurality of specimens; and
(b) the analyzer reading, from a storage stored with data on a known pathway comprising the substances as nodes, the data on the known pathway and determining consistency between the known pathway and the data on the amount of reaction, wherein when the known pathway is an undirected graph, the consistency with the known pathway is determined by the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs; and
(b-5) determining, as a graph consistency probability, a probability density for a range above the combined probability value determined in the above-described step (b-2) in the probability distribution determined in the above-described step (b-4).

An analysis method of another aspect of the invention is for an analyzer to determine a target substance based on data on the amount of reaction obtained by a predetermined treatment performed on a plurality of substances included in a specimen, and the analysis method comprises the steps of:
(a) the analyzer acquiring the data on the amount of reaction for a plurality of specimens;
(b) the analyzer reading, from a storage stored with data on a known pathway comprising the substances as nodes, the data on the known pathway and choosing the known pathway if the known pathway's consistency with the data on the amount of reaction is greater than or equal to a predetermined threshold, wherein when the known pathway stored in the storage is an undirected graph, the known pathway is chosen by the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs;
(b-5) determining, as a graph consistency probability, a probability density for a range above the combined probability value determined in the above-described step (b-2) in the probability distribution determined in the above-described step (b-4); and
(b-6) choosing the known pathway as a pathway consistent with the data on the amount of reaction if the graph consistency probability is less than or equal to a predetermined threshold;
(c) the analyzer determining partial correlation coefficients between the substances based on the data on the amount of reaction of the substances and generating a network structure comprising the substances as nodes based on the partial correlation coefficients; and
(d) the analyzer searching the known pathway chosen in the step (b) and the network structure generated in the step (c) for a same pair of nodes linked to each other and determining substances of found nodes as target substances.

The analysis method of the invention may further comprise the step of:
(e) the analyzer determining, as a signature substance, a substance whose data on the amount of reaction has a difference greater than or equal to a predetermined threshold from the amount of reaction of a control, where also data on a signature substance determined in the above-described step (e) may be used to determine a target substance in the above-described step (d).

### Advantage of the Invention

The invention allows a known pathway's consistency with data on the amount of reaction to be appropriately determined even when the known pathway is an undirected graph.

### BRIEF DESCRIPTION OF THE DRAWINGS

FIG. 1 shows a configuration of an analyzer of a first embodiment;

FIG. 2A shows an example of a known pathway;

FIG. 2B shows an example of the known pathway divided into subgraphs;

FIG. 3A shows an example of data on the amount of reaction to be analyzed;

FIG. 3B shows an example of the correlation between two substances A and B;

FIG. 4 is a table including examples of correlation coefficients between substances;

FIG. 5A shows an example of randomly generated multiple networks;

FIG. 5B shows an example of randomly generated multiple networks;

FIG. 5C shows an example of randomly generated multiple networks;

FIG. 6 shows a probability distribution of combined probabilities of random networks;

FIG. 7 shows an operation of the analyzer of the first embodiment;

FIG. 8 shows a probability distribution of combined probabilities determined by using correlation coefficients instead of partial correlation coefficients;

FIG. 9 shows an overview of an analysis method of a second embodiment;

FIG. 10 shows a configuration of an analyzer of the second embodiment;

FIG. 11 shows an example of network inference;

FIG. 12A shows an example of a known pathway (DAG);

FIG. 12B shows an example of the known pathway (DAG) divided into subgraphs;

FIG. 13 shows an operation of the analyzer of the second embodiment;

FIG. 14 shows an overview of an analysis method of a third embodiment;

FIG. 15 shows a configuration of an analyzer of the third embodiment; and

FIG. 16 shows an operation of the analyzer of the third embodiment.

### MODES OF EMBODYING THE INVENTION

Analysis methods and devices of embodiments of the invention will now be described with reference to the drawings. The description of those embodiments will be made on methods and devices for analyzing consistency between biological data on the amount of reaction obtained by a predetermined treatment performed on a plurality of substances included in a specimen and a known pathway. What to analyze in the embodiments described below is mentioned here. For example, pathways related to immune systems and diseases are preferably evaluated with undirected graphs, because causal connections in those pathways are poorly understood. Specifically, an example of those is a chain of protein-protein interactions including antigen-antibody reactions. The substances are proteins, and the data on the amount of reaction is the affinity of antigen-antibody interactions or the like, in this case. Those presented above are illustrative only, and the analysis methods of the embodiments can be applied to various substances.

### First Embodiment

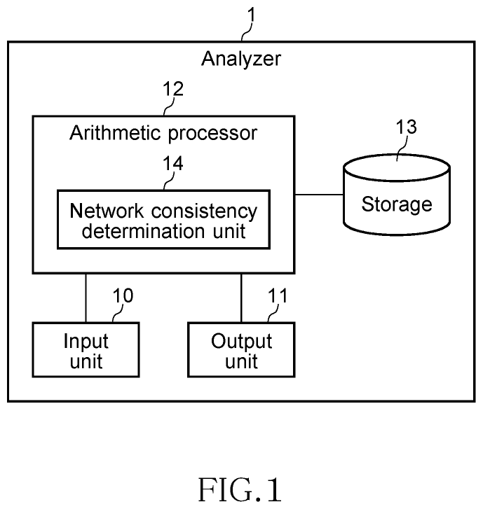

FIG. 1 shows a configuration of an analyzer 1 of a first embodiment. The analyzer 1 of the first embodiment determines consistency between a known pathway having a network structure of an undirected graph and data on the amount of reaction. Undirected graphs to be analyzed by the analyzer 1 of the embodiment are those without a closed circuit. A pathway has a network structure comprising substances as nodes linked to one another, and therefore an evaluation of consistency between a pathway and data on the amount of reaction is made with the pathway being considered as a network. A “pathway” is a connection between substances which is found by experiment, and a “network” is a connection between substances in computational biology. A term “graph” is also used herein, and is a term for mathematically expressing a network structure.

The analyzer 1 comprises an input unit 10 for input of data on the amount of reaction, an output unit 11 for output of an analysis result, and an arithmetic processor 12 for determining consistency between data on the amount of reaction and a known pathway.

The analyzer 1 comprises a computer comprising a CPU, a RAM, a ROM, a hard disk, a display, a keyboard, a mouse, a communications interface, and the like. The computer performs a process of determining consistency between data on the amount of reaction and a known pathway in such a way that a program for an analysis process is stored in the ROM and the CPU reads the program from the ROM and executes it. The program may be stored in a non-volatile storage medium removably mounted on the computer instead of in the ROM. In this case, the computer reads the information processing program from the storage medium and executes it to perform the process of determining consistency between data on the amount of reaction and a known pathway.

An example of the input unit 10 is a communications interface. For example, it receives data on the amount of reaction acquired by a microarray or the like, and puts the data into the analyzer 1. Received data on the amount of reaction is stored on the hard disk for a time. An example of the output unit 11 is a display.

The arithmetic processor 12 will next be described. The arithmetic processor 12 has a network consistency determination unit 14 for determining consistency between inputted data on the amount of reaction and a known pathway. Known pathways in the embodiment are pathways having network structures of undirected graphs without a closed circuit. A process performed by the network consistency determination unit 14 is described as follows.

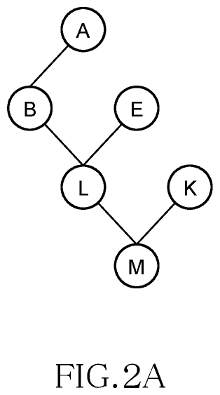

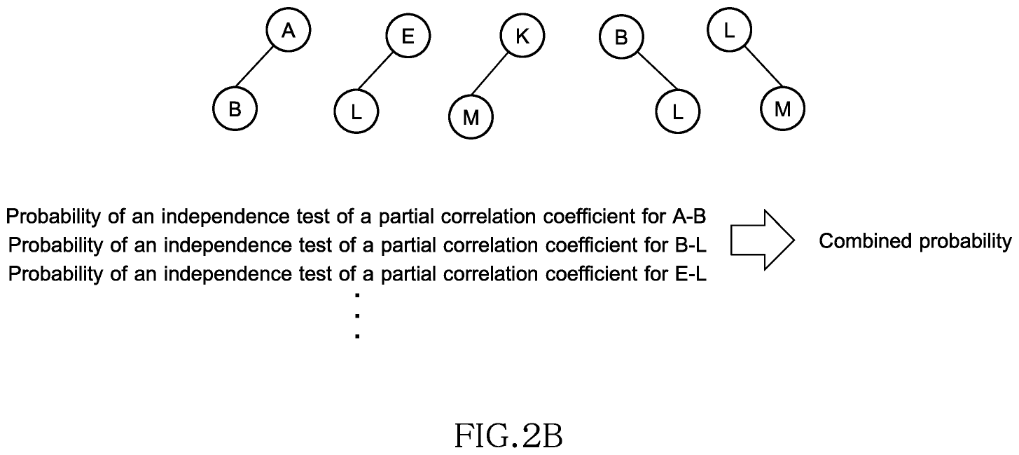

FIG. 2A shows an example of a known pathway. The network consistency determination unit 14 divides the known pathway into subgraphs each comprising two nodes linked to each other as shown in FIG. 2B. The network consistency determination unit 14 determines the first-order partial correlation coefficient between nodes in each subgraph based on data on the amount of reaction. Correlation between substances will be described next.

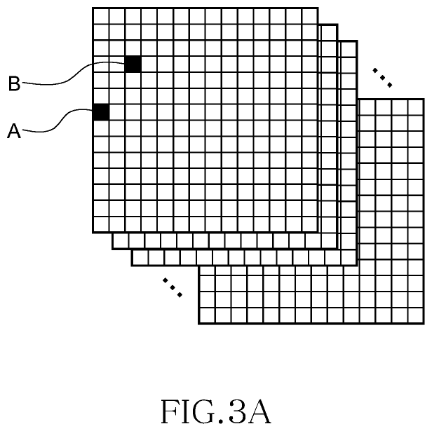

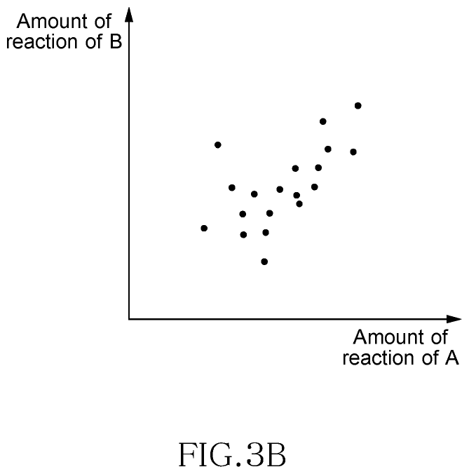

FIG. 3A shows an example of data on the amount of reaction to be analyzed, and expresses data on the amount of reaction in matrices by imitating microarrays. Data on the amount of reaction is quantitative data representing the extent of reaction of each substance caused by a predetermined treatment.

As shown in FIG. 3B, the correlation between two substances A and B, for example, is determined by plotting the amount of reaction of the substance A and that of the substance B in each specimen, so that the correlation coefficient between the two substances can be determined.

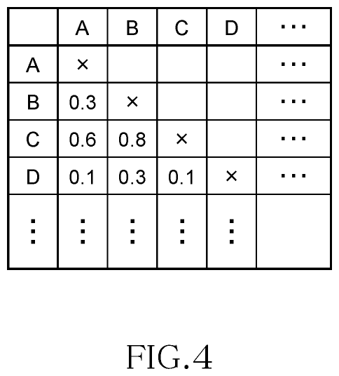

FIG. 4 is a table including examples of correlation coefficients between the substances determined in the manner shown in FIG. 3B. FIG. 4 indicates that the correlation coefficient between the substances A and B is 0.3, and that between the substances A and C is 0.6. First-order partial correlation coefficients between substances are used for evaluating networks in the embodiment. Partial correlation coefficients indicate true correlations excluding the influence of other variables than two targeted variables, and can be calculated by a known method.

The network consistency determination unit 14 uses the partial correlation coefficient between each pair of nodes determined as above to determine the probability of an independence test between each pair of nodes in the known pathway. The network consistency determination unit 14 then uses Fisher's combined probability to integrate the probabilities of the independence tests between the nodes as shown in FIG. 2B, and thereby determines a probability that represents the known pathway's independence from the data on the amount of reaction. The use of partial correlation coefficients in the embodiment allows for detection of spurious correlations, which cannot be detected by using correlation coefficients, and for a more accurate estimation of the whole consistency. While Fisher's combined probability is used in the embodiment to integrate the probabilities of the independence tests between the nodes, another combined probability such as Brown's combined probability can be used for the integration.

The network consistency determination unit 14 then randomly generates many networks each having the same number of nodes and the same number of links as the known pathway. Ten thousand networks are generated in the embodiment. While networks having both the same number of nodes and the same number of links as the known pathway are randomly generated in the embodiment, the number of links does not have to be the same. The network consistency determination unit 14 may randomly generate networks each having the same number of nodes as the known pathway and links whose number is different from that of links in the known pathway.

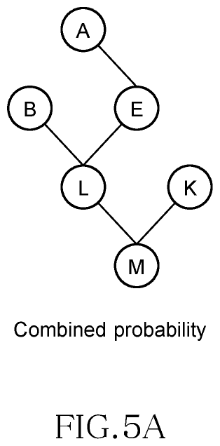

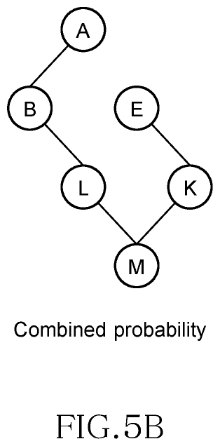

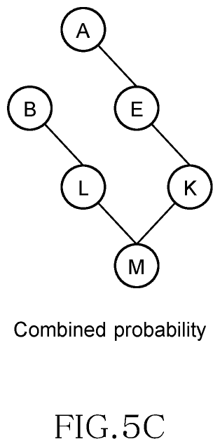

FIGS. 5A to 5C show examples of randomly generated multiple networks. The network consistency determination unit also uses the method described with FIG. 2B to determine the combined probability of each of these networks. Specifically, each network is divided into subgraphs; the probability of an independence test is determined from the partial correlation coefficient of each subgraph; and those probabilities are combined to determine the combined probability value of the network. In this way, combined probabilities are determined for the ten thousand networks.

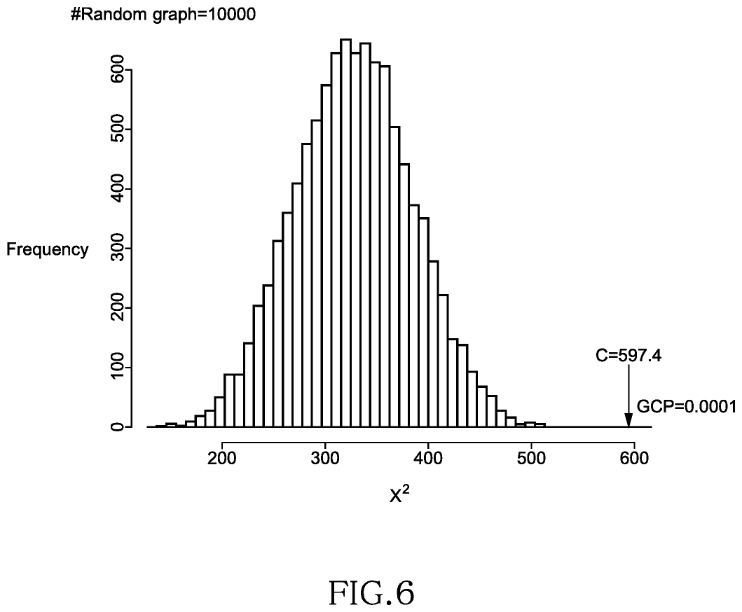

FIG. 6 shows a distribution of the combined probabilities of the randomly generated networks. The horizontal axis of the graph shown in FIG. 6 is the χ² value (chi-square value) of the combined probability of the random networks, and the vertical axis is the frequency. As shown in FIG. 6, the χ² values of the random networks are found to have a distribution whose peak is in between a χ² value of 300 and a χ² value of 360.

A known pathway being consistent with data on the amount of reaction means that the known pathway is not independent of the data on the amount of reaction (a known pathway being independent of data on the amount of reaction means that both are unrelated to each other). For this reason, the χ² value of the combined probability of a known pathway being, for example, in the vicinity of the mean value of the probability distribution of randomly generated networks (within a predetermined range including the mean) means that the known pathway is highly independent of the data on the amount of reaction, and that the consistency is not high.

The network consistency determination unit 14 determines the probability density for a range above the combined probability value of the known pathway in the distribution of the combined probabilities. This probability density is the graph consistency probability (GCP) between the known pathway and the data on the amount of reaction. The GCP getting smaller means that the known pathway is more consistent with the data on the amount of reaction. The network consistency determination unit 14 can determine that there is a consistency with the data on the amount of reaction if the GCP is less than or equal to a predetermined threshold (e.g. 0.2).

The χ² value of the combined probability of the known pathway in the example shown in FIG. 6 is 597.4, and the probability density for a range above this value (+∞) is 0.0001. In other words, the GCP is 0.0001. The GCP, which represents the consistency between a known pathway and data on the amount of reaction, can be determined by the above-described process.

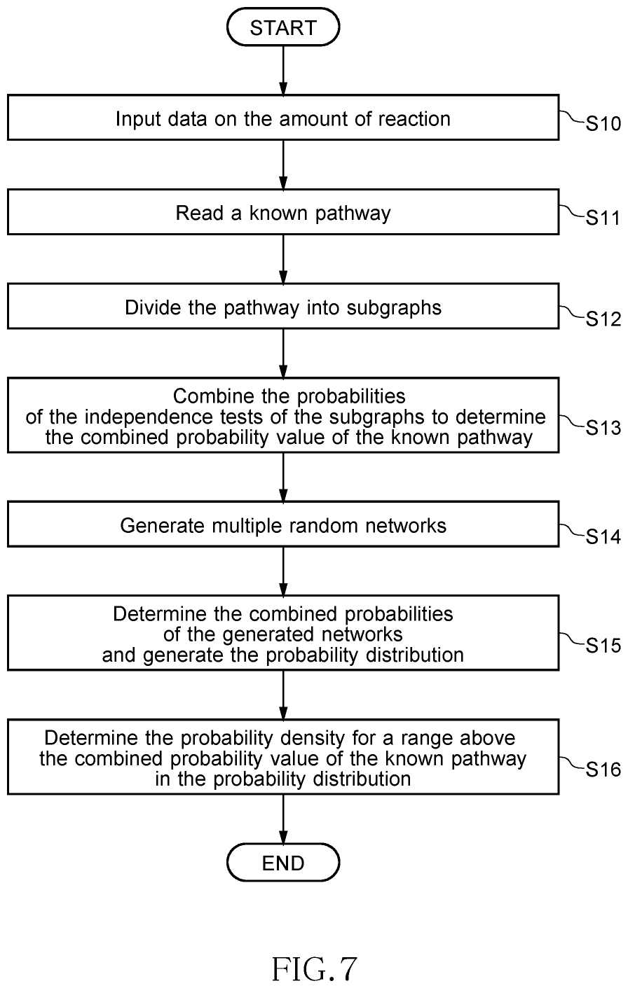

FIG. 7 shows an operation of the analyzer 1 of the first embodiment. The analyzer 1 inputs data on the amount of reaction for a specimen (S10). The analyzer 1 also reads from a storage 13 a known pathway to be compared with the data on the amount of reaction (S11). The analyzer 1 divides the known pathway into a plurality of subgraphs each comprising two nodes connected to each other (S12). The analyzer 1 then determines the probability of the independence test based on the partial correlation coefficient between the nodes of each subgraph, and combines the probabilities to determine the combined probability of the known pathway (S13).

The analyzer 1 then randomly generates networks each having the same number of nodes and the same number of links as the known pathway (S14). Ten thousand networks are generated in the embodiment. Next, the analyzer 1 determines the combined probabilities of the independence tests also for the randomly generated networks, and generates the probability distribution of the combined probabilities (S15). The analyzer 1 determines the probability density for a range above the combined probability value of the known pathway in the generated probability distribution, and sets this probability density as the GCP (S16). The analyzer 1 outputs the value of the determined GCP.

The above is a description of the configuration and operation of the analyzer 1 of the first embodiment. The analyzer 1 of the first embodiment would use the first-order partial correlation coefficient to determine the probabilities of the independence tests between the nodes, and combine thus determined probabilities between the nodes to determine the combined probabilities of the networks. The analyzer 1 would then calculate a GCP based on these combined probabilities, so that an appropriate GCP could be determined and the consistency of the network could be evaluated.

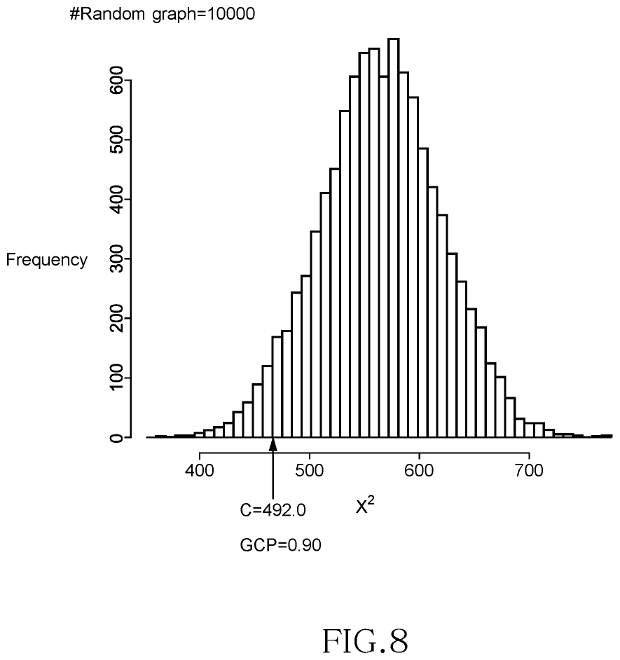

FIG. 8 is an example in which a GCP was determined in the same way as the embodiment for the same data as shown in FIG. 6 by using correlation coefficients instead of first-order partial correlation coefficients. The distribution of the χ² values of combined probabilities had a peak almost in between a χ² value of 550 and a χ² value of 600. The combined probability value of the independence tests for the known pathway was C=492.0, and the probability density for a range above this value (+∞), the GCP, was 0.90. In this case where correlation coefficients were used, it was determined that the known pathway was not consistent with the data on the amount of reaction (was included in the distribution of the randomly generated networks).

Since there was a consistency between the data on the amount of reaction and the known pathway used in the experiment of FIGS. 6 and 8, the determination result for when the partial correlation coefficients were not used is incorrect. It is shown that a correct evaluation can be made by using partial correlation coefficients to evaluate consistency between data on the amount of reaction and a known pathway as in the embodiment.

The analyzer, analysis method, and program stored in a non-volatile storage medium of the embodiment are useful to find a pathway which is activated or whose activation is suppressed in a patient having a predetermined disease. For example, a pathway whose activation is caused or suppressed in a patient having a predetermined disease can be found by inputting data on the difference in the amount of reaction between a patient having a predetermined disease and a healthy person to the analyzer, and searching for a pathway consistent with the data on the amount of reaction. The analyzer, analysis method, and program stored in a non-volatile storage medium of the embodiment are also useful to find a pathway whose activation is caused or suppressed by drug administration. For example, a pathway whose activation is caused or suppressed by a drug can be found by inputting data on the difference in the amount of reaction between before and after drug administration to the analyzer, and searching for a pathway consistent with the data on the amount of reaction. An mRNA expression level, the expression level of protein determined by mass spectrometry, or the like can be used as the amount of reaction. Analyses like this can contribute to drug development.

### Second Embodiment

An analysis method of a second embodiment is a method for determining a target substance based on inputted data on the amount of reaction. A “target substance” is a substance (gene, protein, or the like) that affords the key to identify a disease or the like, or that is expected to afford the key to develop a therapeutic drug for a disease. The search for a target substance considered important is made in this embodiment not only by examining the difference from a control but also by considering a pathway.

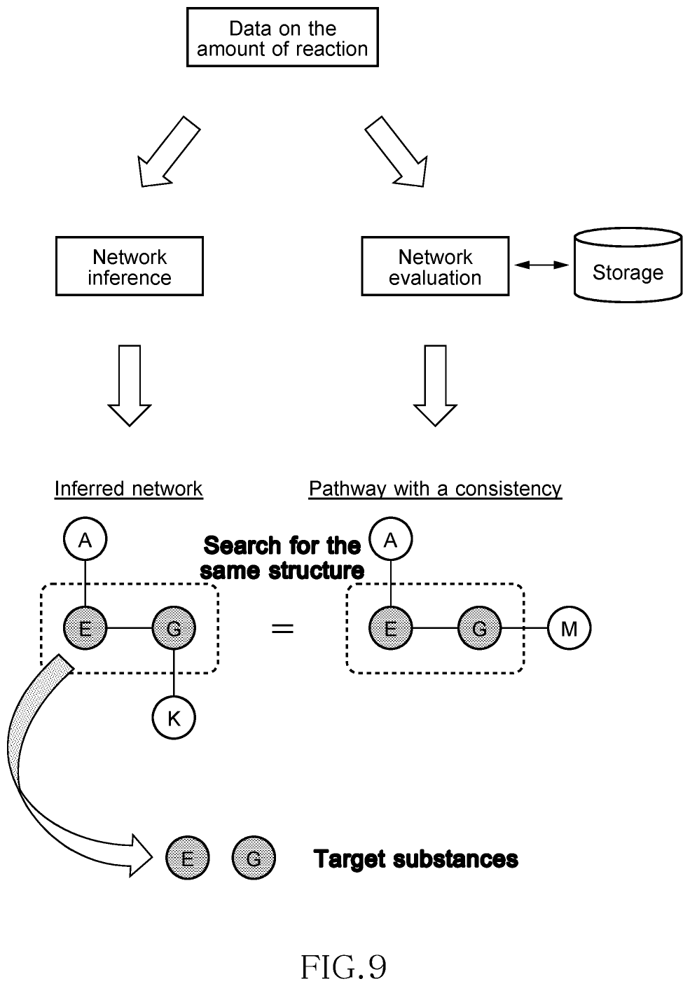

An overview of a process of the analysis method of the second embodiment is described first with reference to FIG. 9. When data on the amount of reaction acquired by a microarray, for example, is inputted, networks of substances are determined in two ways based on the data on the amount of reaction in the analysis method of the second embodiment.

The first way involves evaluating consistency between known pathways and data on the amount of reaction and extracting a highly consistent pathway from among the known pathways. Specifically, an analyzer 2 uses the method described in the first embodiment to determine the GCP between data on the amount of reaction and a known pathway, and determines that there is a consistency between the data on the amount of reaction and the pathway if the GCP is less than or equal to a predetermined threshold. The second way involves inferring a network in which relations between the substances are graphically expressed based on data on the amount of reaction of the substances. How to infer the network will be described later.

The analysis method of the second embodiment comprises detecting data on two nodes that overlap between the pathway evaluated to have a consistency and the inferred network. Nodes E and G are detected in the example shown in FIG. 9 as two nodes that overlap between the two networks. In this way, two substances deeply connected to the pathway can be detected as target substances in the second embodiment.

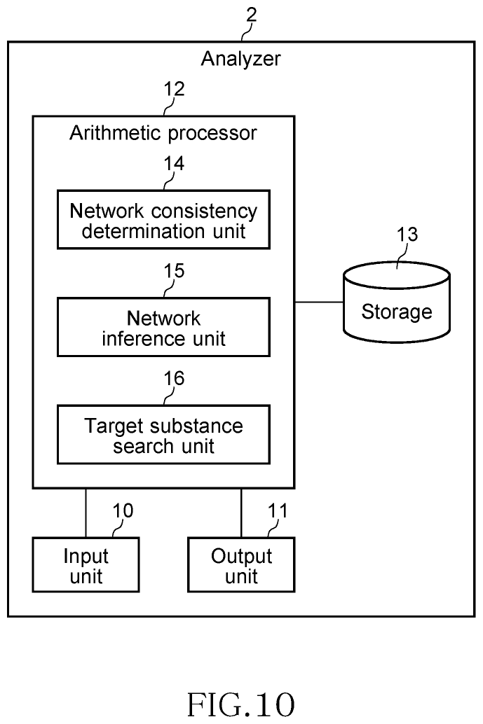

FIG. 10 shows a configuration of the analyzer 2 of the second embodiment. The analyzer 2 comprises an input unit 10 for input of data on the amount of reaction, an output unit 11 for output of an analysis result, and an arithmetic processor 12 for determining a target substance based on data on the amount of reaction.

The analyzer 2 comprises a computer comprising a CPU, a RAM, a ROM, a hard disk, a display, a keyboard, a mouse, a communications interface, and the like. The computer performs a process of analyzing data on the amount of reaction to search for a target substance in such a way that a program for an analysis process is stored in the ROM and the CPU reads the program from the ROM and executes it.

An example of the input unit 10 is a communications interface. For example, it receives data on the amount of reaction acquired by a microarray, and puts it into the analyzer 2. Received data on the amount of reaction is stored on the hard disk for a time. An example of the output unit 11 is a display.

The arithmetic processor 12 will next be described. The arithmetic processor 12 has a network consistency determination unit 14, a network inference unit 15, and a target substance search unit 16. The network inference unit 15 has a function to infer a network comprising substances as nodes based on data on the amount of reaction. The network inference unit 15 determines the partial correlation coefficients between substances based on the amount of reaction of multiple substances included in data on the amount of reaction.

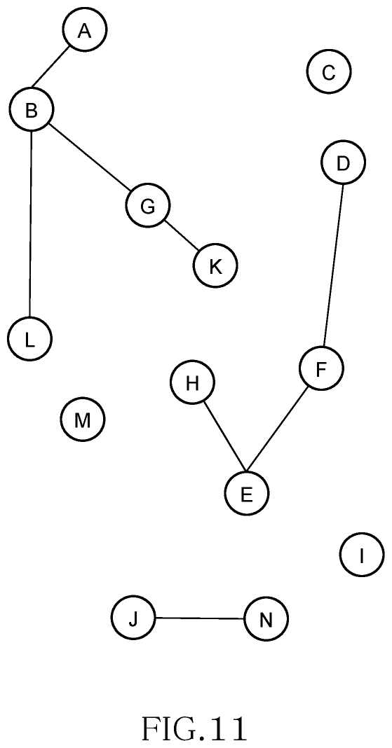

The network inference unit 15 generates links between nodes whose partial correlation coefficients are greater than or equal to a predetermined threshold based on the partial correlation coefficients between substances, thereby generating a network of substances included in data on the amount of reaction. FIG. 11 shows an example of networks inferred by the network inference unit 15. Data on the amount of reaction actually includes more substances than shown in the figure.

The network consistency determination unit 14 has a function to determine consistency between inputted data on the amount of reaction and a known pathway and determine a pathway having a consistency greater than or equal to a predetermined threshold. The network consistency determination unit 14 sequentially reads data on known pathways from the storage 13 stored with known pathways to determine whether they have a consistency with data on the amount of reaction or not. Since a description of when known pathways are undirected graphs is given above, a process for when known pathways are DAGs is described here.

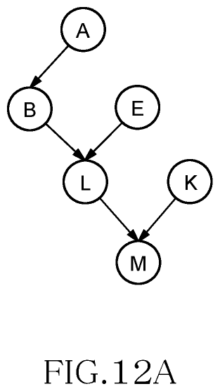

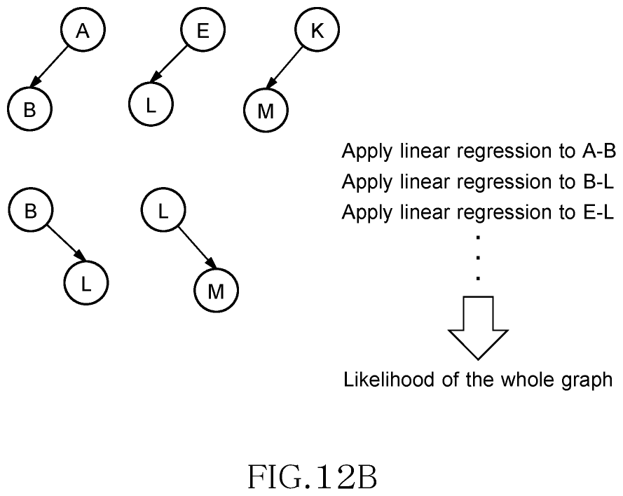

A process for when known pathways are DAGs is described next. FIGS. 12A and 12B show a process of determining the likelihood of the whole graph of a known pathway when it is a DAG. FIG. 12A shows an example of a known pathway. The network consistency determination unit 14 divides the DAG pathway into a plurality of subgraphs each comprising two nodes connected to each other with a conditional probability as shown in FIG. 12B. The network consistency determination unit 14 applies linear regression to each subgraph, and calculates the likelihood of the whole pathway.

The network consistency determination unit 14 also generates a plurality of DAGs each having the same number of nodes and links as the known pathway, and determines the likelihood of each DAG to generate a probability distribution. How to use the probability distribution to determine a GCP is the same as in the case of non-DAGs. The above process allows the GCP between data on the amount of reaction and a known pathway to be determined even if the known pathway is a DAG. The network consistency determination unit 14 can determine a pathway having a consistency with the data on the amount of reaction based on the GCP.

The target substance search unit 16 searches for a structure of two nodes common to both an inferred network and a network having a consistency, and identifies found substances as target substances.

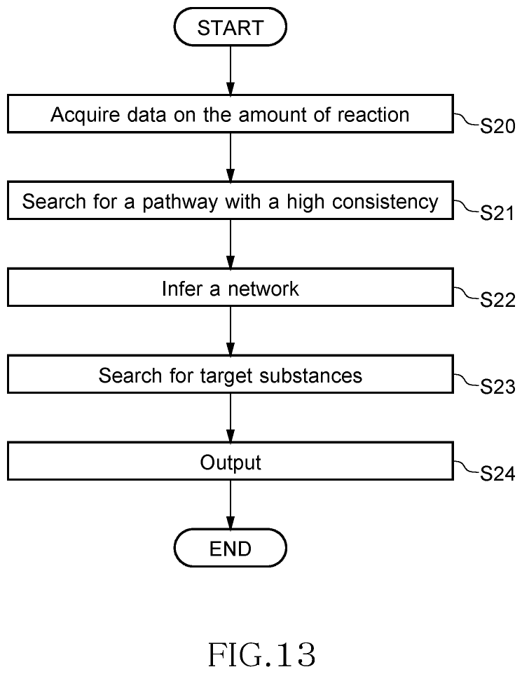

FIG. 13 shows an operation of the analyzer 2 of the second embodiment. Upon acquiring data on the amount of reaction (S20), the analyzer 2 sequentially reads data on known pathways from the storage 13, determines their consistency with the data on the amount of reaction, and determines a pathway with a high consistency (S21). The analyzer 2 also infers a network of substances based on data on the amount of reaction of each substance (S22). The analyzer 2 searches for two nodes commonly included in both the known pathway with a high consistency and the inferred network, and identifies substances of found nodes as target substances (S23). The analyzer 2 outputs information on the identified target substances (S24).

The above is a description of the configuration and operation of the analyzer 2 of the second embodiment. The analyzer 2 of the second embodiment searches for two nodes commonly present in both a network generated from data on the amount of reaction and a known pathway having a consistency with the data on the amount of reaction, and can therefore determine substances that accurately identify a pathway.

Since the analyzer 2 of the embodiment searches for substances using data on known pathways instead of comparing phosphorylation patterns of normal cells and those of specimen cells like the aforementioned prior art, the search for target substances can be made appropriately based on all the pathways.

The analyzer, analysis method, and program stored in a non-volatile storage medium of the embodiment are useful to find a target substance which is activated or whose activation is suppressed in a patient having a predetermined disease. The analyzer and others of the embodiment are also useful to find a target substance whose activation is caused or suppressed by drug administration. While elemental substances would be searched for on the molecular level in the conventional search for a target substance, a target substance is determined in the embodiment by focusing on a pathway that indicates relations between a plurality of substances. This allows a determined target substance to be consistent with a reaction in an organism, so that an appropriate target substance can be found. Analyses like this can contribute to drug development.

### Third Embodiment

An analysis method of a third embodiment narrows down potential target substances using yet another approach in addition to the analysis method of the second embodiment. Specifically, a signature substance, a substance greater in the amount of reaction than a control, is chosen from substances included in data on the amount of reaction, and a target substance is determined also in terms of whether it is a signature substance or not.

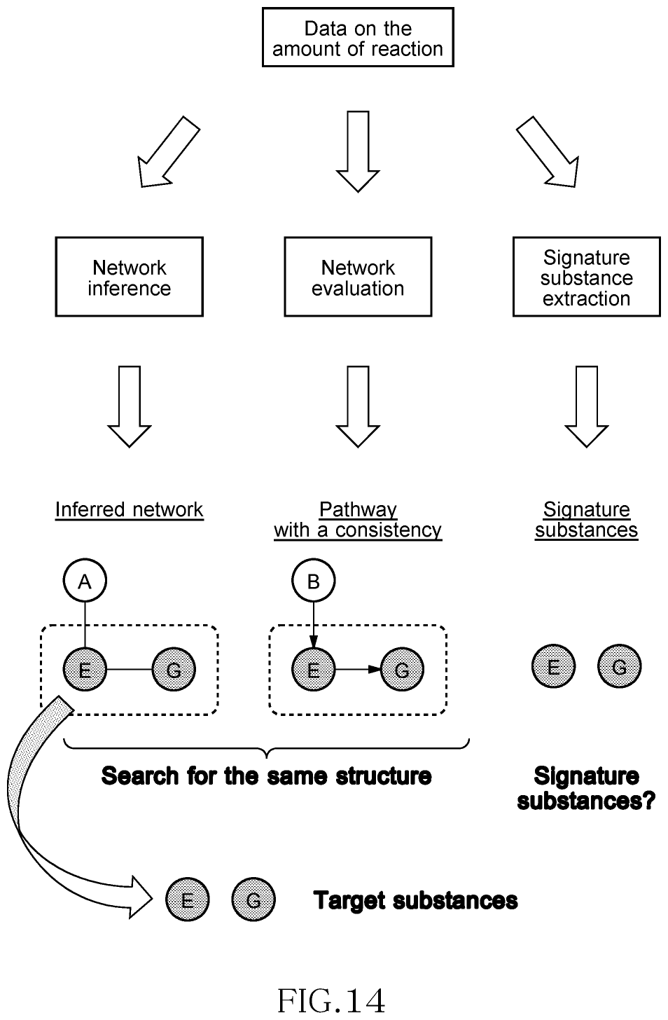

As shown in FIG. 14, the analysis method of the third embodiment comprises detecting data on two nodes that overlap between a pathway evaluated to have a consistency and an inferred network. Whether substances related to the two detected nodes are signature substances or not is then determined and, if they are determined to be signature substances, substances related to Nodes E and G are detected as target substances.

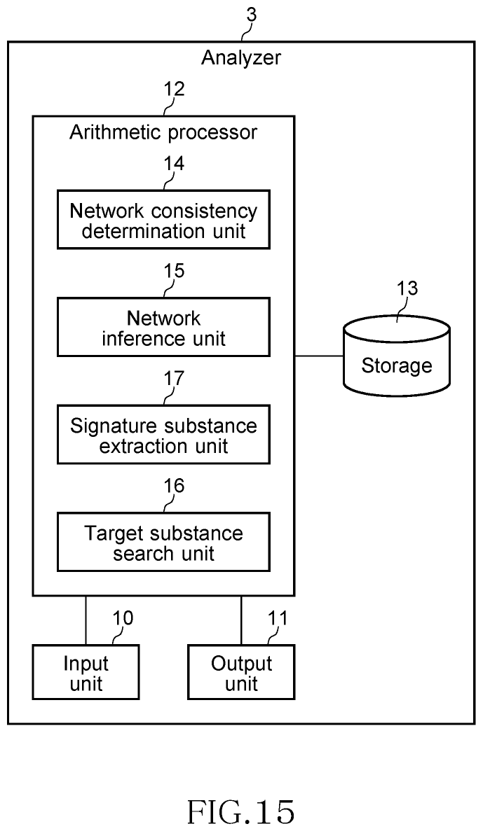

FIG. 15 shows a configuration of an analyzer 3 of the third embodiment. The analyzer 3 of the third embodiment is basically the same in configuration as the analyzer 2 of the second embodiment, but has a signature substance extraction unit 17 in addition to the configuration of the analyzer 2.

The signature substance extraction unit 17 compares inputted data on the amount of reaction of a specimen with data on the amount of reaction of a control, and determines a signature substance, a substance whose amount of reaction is greater than a predetermined threshold as compared with the control. The signature substance extraction unit 17 determines a substance included in a specimen to be a signature substance if, for example, the difference in data on the amount of reaction between the specimen and a control is greater than or equal to a predetermined threshold. Also a technique disclosed in Japanese Patent Application No. 2014-173382 applied by two of the present inventors may be used for the extraction of a signature substance.

A target substance search unit 16 searches for a structure of two nodes commonly present in a known pathway evaluated to have a consistency by a network consistency determination unit 14 and a network inferred by a network inference unit 15. When common nodes are found, the target substance search unit 16 determines whether substances related to the nodes are signature substances or not and, if they are determined to be signature substances, identifies the substances related to the found nodes as target substances.

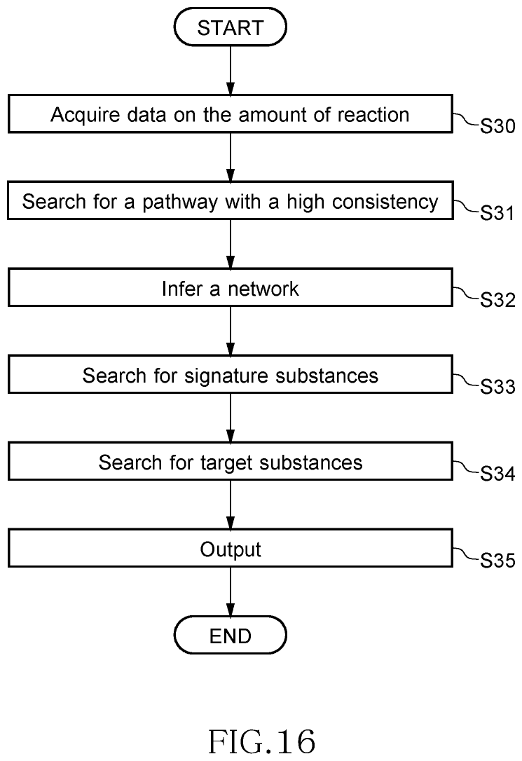

FIG. 16 shows an operation of the analyzer 3 of the third embodiment. Upon acquiring data on the amount of reaction (S30), the analyzer 3 sequentially reads data on known pathways from a storage 13, determines their consistency with the data on the amount of reaction, and determines a network with a high consistency (S31). The analyzer 3 also infers a network of substances based on the amount of reaction of each substance (S32). The analyzer 3 compares inputted data on the amount of reaction of a specimen with data on the amount of reaction of a control, and determines a signature substance (S33).

The analyzer 3 searches for two nodes commonly included in the known pathway with a high consistency and the inferred network, and determines whether substances of found nodes are signature substances or not. If the substances related to the common nodes are signature substances, the analyzer 3 identifies the substances as target substances (S34). The analyzer 3 outputs information on the identified target substances (S35).

The above is a description of the configuration and operation of the analyzer 3 of the third embodiment. As with the second embodiment, the analyzer 3 of the third embodiment can determine substances that accurately identify a pathway.

1. An analysis method for using an analyzer to analyze consistency between data on the amount of reaction obtained by a predetermined treatment performed on a plurality of substances included in a specimen and a known pathway, the analysis method comprising the steps of:
(a) the analyzer acquiring the data on the amount of reaction for a plurality of specimens; and
(b) the analyzer reading, from a storage stored with data on a known pathway comprising the substances as nodes, the data on the known pathway and determining consistency between the known pathway and the data on the amount of reaction, wherein when the known pathway is an undirected graph, the consistency with the known pathway is determined by the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs; and
(b-5) determining, as a graph consistency probability, a probability density for a range above the combined probability value determined in the above-described step (b-2) in the probability distribution determined in the above-described step (b-4).

2. The analysis method according to claim 1, wherein a plurality of graphs each having a same number of nodes and a same number of links as the known pathway are generated in the step (b-3).

3. The analysis method according to claim 1, further comprising the step of:
(b-6) the analyzer determining the known pathway to be consistent with the data on the amount of reaction if the graph consistency probability is less than or equal to a predetermined threshold.

4. An analysis method for an analyzer to determine a target substance based on data on the amount of reaction obtained by a predetermined treatment performed on a plurality of substances included in a specimen, the analysis method comprising the steps of:
(a) the analyzer acquiring the data on the amount of reaction for a plurality of specimens;
(b) the analyzer reading, from a storage stored with data on a known pathway comprising the substances as nodes, the data on the known pathway and choosing the known pathway if the known pathway's consistency with the data on the amount of reaction is greater than or equal to a predetermined threshold, wherein when the known pathway stored in the storage is an undirected graph, the known pathway is chosen by the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs;
(b-5) determining, as a graph consistency probability, a probability density for a range above the combined probability value determined in the above-described step (b-2) in the probability distribution determined in the above-described step (b-4); and
(b-6) choosing the known pathway as a pathway consistent with the data on the amount of reaction if the graph consistency probability is less than or equal to a predetermined threshold;
(c) the analyzer determining partial correlation coefficients between the substances based on the data on the amount of reaction of the substances and generating a network structure comprising the substances as nodes based on the partial correlation coefficients; and
(d) the analyzer searching the known pathway chosen in the step (b) and the network structure generated in the step (c) for a same pair of nodes linked to each other and determining substances of found nodes as target substances.

5. The analysis method according to claim 4, wherein when the known pathway read in the step (b) is a directed acyclic graph, the pathway is chosen by the steps of:
(b-7) dividing the known pathway into a plurality of subgraphs each comprising two nodes connected to each other with a conditional probability;
(b-8) using the data on the amount of reaction to perform linear regression on each subgraph, and determining a likelihood of the whole known pathway;
(b-9) generating a plurality of directed acyclic graphs each having a same number of nodes as the known pathway;
(b-10) determining likelihoods for the plurality of directed acyclic graphs in the same manner as the above-described steps (b-7) and (b-8), and generating a probability distribution of likelihoods of the plurality of directed acyclic graphs;
(b-11) calculating, as a graph consistency probability, a probability density for a range above the likelihood determined in the above-described step (b-8) in the probability distribution determined in the above-described step (b-10); and
(b-12) determining the known pathway to be consistent with the data on the amount of reaction if the graph consistency probability is less than or equal to a predetermined threshold.

6. The analysis method according to claim 4, further comprising the step of:
(e) the analyzer determining, as a signature substance, a substance whose data on the amount of reaction has a difference greater than or equal to a predetermined threshold from the amount of reaction of a control,
wherein also data on a signature substance determined in the above-described step (e) is used to determine a target substance in the above-described step (d).

7. An analyzer for analyzing consistency between data on the amount of reaction obtained by a predetermined treatment performed on a plurality of substances included in a specimen and a known pathway, the analyzer comprising:
an input unit for input of data on the amount of reaction for a plurality of specimens;
a storage stored with data on a known pathway comprising the substances as nodes; and
a network consistency determination unit for reading the data on the known pathway from the storage and determining consistency between the known pathway and the data on the amount of reaction,
wherein when the known pathway is an undirected graph, the network consistency determination unit determines a graph consistency probability that represents consistency between the known pathway and the data on the amount of reaction, using the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs;
(b-5) determining, as a graph consistency probability, a probability density for a range above the combined probability value determined in the above-described step (b-2) in the probability distribution determined in the above-described step (b-4); and
(b-6) choosing the known pathway as a pathway consistent with the data on the amount of reaction if the graph consistency probability is less than or equal to a predetermined threshold.

8. An analyzer for determining a target substance based on data on the amount of reaction obtained by a predetermined treatment performed on a plurality of substances included in a specimen, the analyzer comprising:
an input unit for input of data on the amount of reaction for a plurality of specimens;
a storage stored with data on known pathways each comprising the substances as nodes;
a network consistency determination unit for reading the data on the known pathways from the storage and choosing a pathway from the known pathways that is consistent with the data on the amount of reaction;
a network inference unit for determining partial correlation coefficients between the substances based on the data on the amount of reaction and generating a network structure comprising the substances as nodes based on the partial correlation coefficients; and
a target substance search unit for searching the known pathway chosen by the network consistency determination unit and the network structure generated by the network inference unit for a same pair of nodes linked to each other and determining substances of found nodes as target substances,
wherein when the known pathways stored in the storage are undirected graphs, the network consistency determination unit chooses a known pathway consistent with the data on the amount of reaction using the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs;
(b-5) determining, as a graph consistency probability, a probability density for a range above the combined probability value determined in the above-described step (b-2) in the probability distribution determined in the above-described step (b-4); and
(b-6) choosing the known pathway as a pathway consistent with the data on the amount of reaction if the graph consistency probability is less than or equal to a predetermined threshold.

9. A program for analyzing consistency between data on the amount of reaction obtained by a predetermined treatment performed on a plurality of substances included in a specimen and a known pathway, the program causing a computer to execute the steps of:
(a) acquiring the data on the amount of reaction for a plurality of specimens; and
(b) reading, from a storage stored with data on a known pathway comprising the substances as nodes, the data on the known pathway and determining consistency between the known pathway and the data on the amount of reaction, wherein when the known pathway is an undirected graph, the consistency with the known pathway is determined by the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs;
(b-5) determining, as a graph consistency probability, a probability density for a range above the combined probability value determined in the above-described step (b-2) in the probability distribution determined in the above-described step (b-4); and
(b-6) choosing the known pathway as a pathway consistent with the data on the amount of reaction if the graph consistency probability is less than or equal to a predetermined threshold.

10. A non-volatile storage medium stored with a program for determining a target substance based on data on the amount of reaction obtained by a predetermined treatment performed on a plurality of substances included in a specimen, the non-volatile storage medium causing the program to run and cause a computer to execute the steps of:
(a) acquiring the data on the amount of reaction for a plurality of specimens;
(b) reading, from a storage stored with data on a known pathway comprising the substances as nodes, the data on the known pathway and choosing the known pathway if the known pathway's consistency with the data on the amount of reaction is greater than or equal to a predetermined threshold, wherein when the known pathway stored in the storage is an undirected graph, the known pathway is chosen by the steps of:
(b-1) dividing the known pathway into a plurality of subgraphs each comprising two nodes connected to each other;
(b-2) applying the data on the amount of reaction to each subgraph to determine a partial correlation coefficient between each pair of nodes, and combining probability values of independence tests of the determined partial correlation coefficients to determine a combined probability value that represents the known pathway's independence from the data on the amount of reaction;
(b-3) generating a plurality of graphs each having a same number of nodes as the known pathway;
(b-4) determining combined probabilities for the plurality of graphs in the same manner as the above-described steps (b-1) and (b-2), and generating a probability distribution of combined probability values of the plurality of graphs;
(b-5) determining, as a graph consistency probability, a probability density for a range above the combined probability value determined in the above-described step (b-2) in the probability distribution determined in the above-described step (b-4); and
(b-6) choosing the known pathway as a pathway consistent with the data on the amount of reaction if the graph consistency probability is less than or equal to a predetermined threshold;
(c) determining partial correlation coefficients between the substances based on the data on the amount of reaction of the substances and generating a network structure comprising the substances as nodes based on the partial correlation coefficients; and
(d) searching the known pathway chosen in the step (b) and the network structure generated in the step (c) for a same pair of nodes linked to each other and determining substances of found nodes as target substances.
